(19) United States
(12) Patent Application Publication
(10) Pub. No.: US 2012/0052513 A1
(43) Pub. Date: Mar. 1, 2012

(54) GOLD SUB-NANOCLUSTERS AND USES THEREOF

(76) Inventors: Pradeep THALAPPIL, Chennai (IN); Madathumpady Abubaker Habeeb Muhammed, Kerala (IN)

(21) Appl. No.: 12/862,480

(22) Filed: Aug. 24, 2010

(51) Int. Cl. G01N 33/574; C07F 1/12; C07D 311/78; C03C 25/38; C07H 21/00; C07K 16/00; G01N 21/76; G01N 33/20; C07F 7/00; C07K 14/00

(52) U.S. Cl. 435/7.23; 556/9; 556/113; 549/212; 530/400; 556/13; 536/22.1; 530/391.1; 436/172; 436/80; 216/83; 977/700

(57) ABSTRACT

The present technology relates gold sub-nanoclusters comprising a gold core and one or more thiolates bound to the gold core, wherein the gold core consists essentially of 23 gold atoms. Methods of preparing and using such sub-nanoclusters are also provided.

### BACKGROUND

Sub-nanoclusters of noble metals differ substantially from metallic nanoparticles of the same element in both structure and physical properties. While metal nanoparticles may range from a few nanometers (nm) to hundreds of nm in size, sub-nanoclusters have dimensions of about 1 nm or less and include relatively few atoms of metal. Due to the sub-nanometer core size, sub-nanoclusters cannot possess continuous density of states but have discrete electronic energy levels. They show “molecule-like” optical transitions in absorption and emission and therefore show characteristic features different not only from those of nanoparticles but from individual atoms as well.

The absorption profiles of sub-nanoclusters of various sizes can be distinguished from each other and from nanoparticles. Sub-nanoclusters exhibit strong photoluminescence and their luminescence ranges from the near-infrared (NIR) region to the ultraviolet as core size decreases. While metal nanoparticles of 2-3 nm exhibit very weak luminescence with quantum yields of about 10⁻⁴ to 10⁻⁵, sub-nanoclusters can exhibit luminescence with quantum yields in the range of 10⁻¹ to 10⁻³. Moreover, sub-nanoclusters may be readily conjugated to biological molecules and their low metal content makes them more biocompatible than nanoparticles. Such properties allow for the use of sub-nanoclusters in imaging and detection, especially medical imaging and in conjunction with therapeutics.

Discovery and use of metal sub-nanoclusters remain an empirical enterprise. While current techniques have allowed for the preparation of a variety of sizes of sub-nanoclusters, not all sizes have proven accessible. Some sub-nanoclusters are thermodynamically unstable and luminescence quantum yields vary between sub-nanoclusters. In addition, methods such as the reduction of Au³⁺ ions in the presence of glutathione provide a complex mixture of sub-nanoclusters that must be separated by, e.g., polyacrylamide gel electrophoresis (PAGE).

### SUMMARY

The present technology provides gold sub-nanoclusters with good thermodynamic stability and luminescence quantum yields and reliable methods of making such sub-nanoclusters. The present technology further provides conjugates and compositions including such sub-nanoclusters and methods of using the same.

In one aspect, the present technology provides gold sub-nanoclusters including a gold core and one or more thiolates bound to the gold core. In some embodiments, the gold core includes or consists essentially of 23 gold atoms. In some embodiments, the one or more thiolates are selected from glutathione thiolate, 3-mercaptopropyl trimethoxy silane, octanethiolate and a mixture of any two or more thereof. In some embodiments, the one or more thiolates is glutathione thiolate. In an illustrative embodiment, the gold sub-nanocluster has the formula Au₂₃(SG)₁₈ wherein SG is glutathione thiolate.

In some embodiments of the present technology, the gold sub-nanoclusters further include one or more amine or phosphine ligands. In some embodiments of the present technology, the gold sub-nanoclusters further include one or more fluorescent ligands selected from dansyl, fluorescein isothiocyanate (FITC), green fluorescent protein, coumarin, fluorescein, and cyanine dyes.

In other embodiments, the gold sub-nanocluster of the present technology may be conjugated to a targeting molecule selected from the group consisting of a protein, a polynucleotide, or a ligand that binds to a protein or polynucleotide. Illustrative targeting molecules include but are not limited to streptavidin, biotin, an antibody, folic acid, lactoferrin, transferrin, or tat protein.

In another aspect, the present technology provides a composition including an aqueous solution, an organic solution or a mixture thereof that includes one or more gold sub-nanoclusters as described herein.

In yet another aspect, the present technology provides methods of making gold sub-nanoclusters. The methods include core etching of one or more Au₂₅SG₁₈ sub-nanoclusters using a molar excess of thiolate relative to the molar amount of SG, to provide one or more gold sub-nanoclusters, wherein SG is glutathione thiolate. In some embodiments of the methods, the one or more Au₂₅SG₁₈ sub-nanoclusters are dissolved in an aqueous solution and the excess thiolates are dissolved in an organic solvent that forms a two-phase system with the aqueous solution. The organic solvent may, e.g., be selected from toluene and xylene. In some embodiments of the methods, the thiolate may be selected from glutathione thiolate, octanethiolate, 3-mercaptopropyl trimethoxy silane thiolate and a mixture of any two or more thereof.

Various gold nanoclusters may be produced by the present methods. In some embodiments of the methods, the gold sub-nanocluster produced consists essentially of 23 atoms of gold and one or more thiolates such as, e.g., glutathione thiolate. In some embodiments, the gold sub-nanocluster produced has the formula Au₂₃SG₁₈ (wherein SG is glutathione thiolate). In other embodiments, the gold sub-nanocluster produced contains 22 or 33 gold atoms. In some embodiments, the gold sub-nanoclusters produced have the formula Au₂₂(MPTS)₁₀(SG)₇ or Au₃₃OT₂₂.

In another aspect, there are provided methods of using the sub-nanoclusters of the present technology. The methods include labeling a biological target with a conjugate of a gold sub-nanocluster as described herein, and detecting the conjugate by luminescence. In some embodiments of the present methods, the biological target may be a cancer cell. In other embodiments, the luminescence may be excited at a wavelength from about 400 to about 550 nm and the emission may be detected at a wavelength from about 600 nm to about 800 nm.

In yet another aspect of the present technology, there are provided methods of detecting Cu²⁺ ions in an aqueous sample. The methods include treating an aqueous sample to be tested for Cu²⁺ ions with an effective amount of gold sub-nanoclusters and measuring the decrease in fluorescence of the treated sample, wherein the gold sub-nanocluster includes a gold core and one or more thiolates bound to the gold core, and wherein the gold core consists essentially of 23 gold atoms.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1A-C show mass spectra of an illustrative embodiment of the present technology: A) MALDI-MS of the gold sub-nanoclusters showing peaks due to gold thiolate clusters (in which only AuₘSₙ clusters are visible since the laser irradiation cleaves the S—C bond of thiolate ligands); B) a group of peaks with m/z spacing of 197 or 229 between the major peaks of the adjacent group of peaks; and C) expanded view of peaks due to Au₂₃S₁₈₋₂₃.

FIG. 2A-D show Fourier Transform Infrared (FTIR) spectra of illustrative embodiments of the starting material, Au₂₅(SG)₁₈, as well as the gold sub-nanoclusters of the present technology and the corresponding thiols used for etching,

FIG. 3A-B show inherent luminescence of illustrative embodiments of subnanoclusters of the present technology, including (A) Au₂₂ and (B) Au₂₃ collected by spectroscopic mapping at an excitation wavelength of 532 nm. Light regions represent the pixels where the signal (used for the mapping) is at a maximum, the minima being represented with black. The scan area was 40 μm×40 μm.

FIG. 4A-C show an illustrative embodiment of (A) fluorescent, (B) bright field, and (C) overlay of fluorescent and bright field images of human hepatoma cells stained with streptavidin-conjugated Au₂₃.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

The present technology provides gold sub-nanoclusters, compositions and conjugates of such sub-nanoclusters and methods of making and using the same. Such sub-nanoclusters are luminescent, e.g., fluorescent in the near infrared, may be coated with thiolate, are relatively non-toxic and are therefore useful in various imaging applications, including, e.g., fluorescent patterns for soft lithography, protein chips, medical imaging and in conjunction with therapy. In particular, such sub-nanoclusters may be derivatized with ligands that selectively bind to biological targets and allow them to be used as fluorescent labels for the biological targets.

In one aspect, the present technology provides gold sub-nanoclusters including a gold core and one or more thiolates bound to the gold core. The gold core refers to the gold atoms in the sub-nanocluster, but does not include any surface modifications of the gold atoms such as thiolates or other molecules which are bound to the surfaces of the gold atoms. Gold sub-nanoclusters of the present technology include Au₂₂, Au₂₃, and Au₃₃ cores. The luminescence quantum yields of the Au₂₂ and Au₂₃ cores are 2.5% and 1.3%, respectively, making them significantly brighter fluorescent labels than the similar Au₂₅ cores (about 0.1% quantum yield).

Gold sub-nanoclusters of the present technology may include thiolates bound to the gold core. A thiolate is the anionic form (—S⁻) of the chemical group, thiol (—SH). Both water soluble and non-water soluble thiolates may be used. Thiolates that may be bound to the gold core include but are not limited to glutathione thiolate (SG), 3-mercaptopropyl trimethoxy silane (MPTS), octanethiolate (OT) and a mixture of any two or more thereof. In some embodiments, the gold sub-nanoclusters include, consist of or consist essentially of 23 gold atoms and glutathione thiolate, for example, Au₂₃(SG)₁₈. Other illustrative embodiments include but are not limited to Au₂₂(MPTS)₁₀(SG)₇ and Au₃₃(OT)₂₂.

In still other embodiments, amine and phosphine ligands may be bound to the gold core in addition to or in place of thiolates through, e.g., interfacial ligand exchange involving two or more immiscible phases such as two immiscible liquids or solid-liquid phases, and the like. For example, alkyl amines including but not limited to 1-butylamine, 1-hexylamine, and 1-octylamine as well as amino acids with amine side-chains such as lysine or ornithine may be exchanged onto the surface of gold sub-nanoclusters of the present technology. Likewise, phosphines including but not limited to triphenylphosphines, salts of sulfonated phosphines (e.g., 4-(diphenylphosphino)benzenesulfonic acid), and the like.

Gold sub-nanoclusters of the present technology may also include one or more fluorescent ligands. Suitable ligands include but are not limited to dansyl, fluorescein isothiocyanate (FITC), green fluorescent protein, coumarin, fluorescein, and cyanine dyes. In illustrative embodiments, ligands include but are not limited to acridine, 7-amino-4-methyl coumarin-3-acetic acid (AMCA), boron dipyrrolemethene (BODIPY), Cascade Blue, Cy2, Cy3, Cy5, Cy7, Edans, Eosin, Erythrosin, 6-Fam, Tet, Joe, Hex, Oregon Green, Rhodamine, Rhodol Green, Tamra, Rox, and Texas Red.

Such fluorescent ligands may be readily attached to suitably functionalized thiolates. By way of non-limiting example, fluorescent ligands containing amino or carboxyl groups may be coupled to a thiolate bearing a carboxyl or amino group, respectively, using standard procedures for amide bond formation (see, e.g., S—Y. Han and Y-A. Kim, Recent development of peptide coupling reagents in organic synthesis. Tetrahedron, 2004, 60, 2447). Thus, coupling agents (e.g., EDC), active esters (e.g., pentafluorophenol), mixed anhydrides and the like may all be used to form amide bonds between, e.g., a carboxyl-bearing thiolate of the sub-nanocluster and an amino-bearing ligand. Other types of linkages such as urethane and thiourea may also be formed from, e.g., isocyanates with amines or thiols. Similarly, click chemistry such as, e.g., the copper catalyzed Huisgen azide-alkyne reaction, may be used to attach fluorescent ligands to suitable functional groups on the thiolates. Depending on the type of chemistry, the reactions may be carried out directly on the sub-nanoclusters, or thiolates of the sub-nanoclusters may be exchanged for fluorescent-containing thiolates prepared according to the reactions described above.

Gold sub-nanoclusters may be conjugates that include a targeting molecule. By “targeting molecule” is meant a molecule that specifically binds to another molecule, i.e. has a binding constant with its partner of about 10 μM or less, and in some embodiments, 1 μM or less, 0.1 μM or less, or even 0.01 μM or less. A targeting molecule may be a protein, a polynucleotide, or a ligand that binds to a protein or polynucleotide. Such ligands include not only large biological macromolecules, such as but not limited to antibodies, receptors, enzymes, oligonucleic acids, and aptamers, but small organic molecules, such as but not limited to folic acid and cofactors, such as but not limited to, biotin. Thus, in illustrative embodiments, targeting molecules include but are not limited to streptavidin, biotin, antibodies, folic acid, lactoferrin, transferrin, or tat protein.

Such conjugates may be prepared by coupling a suitably functionalized thiolate to the targeting molecule in much the same way that fluorescent ligands may be attached to thiolates as described above. For example, antibodies, other proteins and small molecules bearing carboxyl or amino groups may be linked, respectively, to the amino or a carboxyl group of glutathione via amide bond formation as described herein for fluorescent ligands. Likewise, urethanes, thiourethanes, and thioureas may also be formed from suitably functionalized thiolates and targeting molecules. Click chemistry may also be employed to prepare such conjugates.

In one aspect, the present technology provides compositions including aqueous solutions, organic solutions or mixtures thereof that include one or more gold sub-nanoclusters described herein. Such compositions may be useful in imaging applications and may be readily prepared using water or organic solvents as appropriate to the particular application at hand. Aqueous solutions of, e.g., Au₂₃SG₁₈, may be used to stain cells in vitro or in vivo. Organic solutions of, e.g., Au₃₃(OT)₂₂ may be used for catalysis of chemical reactions. Particularly with respect to in vivo use, aqueous solutions may include pharmaceutically acceptable carriers. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

The gold sub-nanoclusters described herein may be prepared by core etching (also known as “interfacial etching”) one or more Au₂₅SG₁₈ sub-nanoclusters using a molar excess of thiol relative to the molar amount of SG. The types of gold sub-nanoclusters that may be made will vary with the conditions used. In some embodiments of the methods, the Au₂₅SG₁₈ sub-nanoclusters are dissolved in an aqueous solution and the excess thiols (which form thiolates) are dissolved in an organic solvent that forms a two-phase system with the aqueous solution. The organic solvent may, e.g., be selected from aromatic solvents such as, but not limited to, toluene and xylene, chlorocarbons, such as but not limited to chloroform and dichloromethane. Various thiols may be used including but not limited to hexane thiol, decane thiol, phenylethane thiol, mercaptopropionic acid, mercaptosuccinic acid, glutathione, octanethiol, 3-mercaptopropyl trimethoxy silane thiol and a mixture of any two or more thereof. The molar excess of thiol may range, e.g., from about 2 to about 4, about 6 or about 8 equivalents per mole of SG in the Au₂₅SG₁₈ sub-nanoclusters. In some embodiments, the excess thiol may range from about 2 to about 100, about 2 to about 50, about 2 to about 30, about 2 to about 15, or about 2 to about 10 equivalents. The core-etching may be carried out at or near room temperature (e.g., about 20° C. to about 30° C., or at about 25° C.) or at an elevated temperature, e.g., about 50° C. to about 60° C. or about 55° C. In general, lower temperatures may be used to produce smaller clusters, while higher temperatures may be used to produce larger clusters. The reaction time may range from a few minutes to a few hours, including but not limited to, about 5 minutes to about 24 hours, about 10 minutes to about 12 hours, about 30 minutes to about 1, 2, 3, 4, 5, or 6 hours.

In other methods, the Au₂₅SG₁₈ sub-nanoclusters are dissolved in aqueous solution. Excess thiol is added (e.g., up to about 4 equivalents or from about 1.5 equivalents to about 3 equivalents, or about 2 equivalents per mole of SG in the Au₂₅SG₁₈ sub-nanoclusters) and the reaction mixture stirred for several hours at or near room temperature (e.g., about 20° C. to about 30° C., or at about 25° C.). The thiol, such as MPTS, should be readily soluble in water. Reaction times similar to those for the two-phase system may be used.

Core-etching methods typically provide predominantly a single species of sub-nanoclusters rather than the spectrum of species that result from some other methods. However, in the event that further purification is needed, PAGE may be used. Purity may also be assessed using PAGE, optical absorption spectroscopy, and/or mass spectrometry.

Various gold nanoclusters may be produced by the present methods. In some embodiments of the methods, the gold sub-nanocluster produced consists essentially of 23 atoms of gold and one or more thiolates such as, e.g., glutathione thiolate. In some embodiments, the gold sub-nanocluster produced has the formula Au₂₃SG₁₈. In other embodiments, the gold sub-nanocluster produced contains 22 or 33 gold atoms. In some embodiments, the gold sub-nanoclusters produced have the formula Au₂₂(MPTS)₁₀(SG)₇ or Au₃₃OT₂₂.

The gold sub-nanoclusters of the present technology may be used for detection and imaging of biological and other targets. The methods include labeling a biological target with a conjugate of a gold sub-nanocluster as described herein, and detecting the conjugate by luminescence including but not limited to fluorescence and infrared. In addition, such conjugates may be detected using one or more of Raman resonance, NMR, EPR, mass and optical spectra. Such conjugates may also be labeled with a radionuclide emitting alpha, beta, and/or gamma particles. Thus, optical, electronic, ionic and radioactive signatures may be used to capture information.

In some embodiments of the present methods, the biological target may be a cancer cell such as, e.g., hepatoma. Many types of cancer cells may be targeted using target molecules of the present technology. For example, cancer cells containing folic acid receptors can be stained using clusters conjugated with folic acid. Thus, in some embodiments, the biological target is ovarian, kidney, brain, lung or breast cancer cells.

Standard fluorescence techniques may be used for detection of the luminescent sub-nanoclusters in the present methods. For example, confocal fluorescence microscopy may be used in vitro to examine a suitable prepared sample. By way of non-limiting example, cells to be examined may be washed free of growth medium, fixed in a paraformaldehyde solution (e.g., 3%) and exposed to a solution of the present gold sub-nanoclusters. After the cells have been stained, they are washed and imaged with the confocal fluorescence microscope. The luminescence may be excited at any suitable wavelength such as one from about 400 to about 550 nm and the emission may be detected at a wavelength from about 600 nm to about 800 nm.

The present technology may also be used for sensing certain types of metal ions in aqueous samples. Such samples may include, e.g., ground water, well water, or wastewater and may be performed in the context of pollution detection or other purposes. In particular, low concentrations of Cu²⁺ in, e.g., the ppm range, may be selectively detected in water versus Ag³⁺, Ag⁺, Ni²⁺, Ca²⁺, Mg²⁺, Na⁺, Pb²⁺, Hg²⁺ and Cd²⁺. When a sample containing Cu²⁺ ions is treated with an effective amount of Au₂₃ sub-nanoclusters of the present technology, the luminescence of the sample is decreased or even quenched. Thus, in some embodiments, a decrease in luminescence (e.g., fluorescence) indicates the presence of Cu²⁺ ions in the sample. By “effective amount of gold sub-nanoclusters” is meant an amount sufficient (in comparison to the amount of Cu²⁺ ions in the sample) to produce a detectable change in luminescence of the sample. In some embodiments, the luminescence of the test sample is compared to that of a control sample. The control sample may include the gold sub-nanoclusters but no Cu²⁺ ions.

Luminescence of gold sub-nanoclusters of the present technology may be further enhanced upon phase transfer (e.g., with tetraoctylammonium bromide) from aqueous solution to an organic solution. The luminescence of the phase transferred cluster in toluene may be further enhanced upon the addition of alcohols. The luminescence intensity was enhanced according to the series: methanol<ethanol<propanol<butanol. Thus, in some embodiments of the methods herein, the methods further include treating an aqueous solution of the gold sub-nanoclusters with a water-immiscible organic solvent (e.g., an aromatic solvent such as toluene, benzene or xylene) and a phase transfer catalyst. The luminescence of such phase-transferred gold sub-nanoclusters may be detected as described herein, optionally in the presence of alcohols, e.g., C₁₋₄ alcohols.

### EXAMPLES

The present technology is further illustrated by the following examples, which should not be construed as limiting in any way.

Materials. Tetrachloroauric acid trihydrate (HAuCl₂.3H₂O) was purchased from CDH, India. Glutathione (GSH), 1-octanethiol (OT), 3-mercaptopropyl trimethoxysilane (MPTS), tetraoctyl ammonium bromide (TOABr), and sodium borohydride (NaBH₄) were purchased from Sigma-Aldrich. Streptavidin was purchased from Hi-Media Chemicals, India. All chemicals were used as such without further purification. Triply distilled water was used throughout the experiments. Solvents were analytical grade.

### Example 1

Synthesis of glutathione-capped gold (AuSG) clusters. Glutathione capped gold clusters were synthesized according to a reported method. (Negishi, Y., et al. J. Am. Chem. Soc. (2005) 127, 5261, the entire contents of which are incorporated herein by reference.) Reduced glutathione (GSH; 20 mM) was added to a solution of HAuCl₄.3H₂O (100 mL, 5 mM) in methanol. The mixture was then cooled to 0° C. in an ice bath for 30 minutes (min). An aqueous solution of NaBH₄ (25 mL, 0.2 M), cooled at 0° C., was injected rapidly into this mixture under vigorous stirring. The mixture was allowed to react for another hour. The resulting precipitate was collected and washed repeatedly with methanol/water (3:1) through centrifugal precipitation and dried to obtain the Au@SG clusters as a dark brown powder. This product is a mixture of small nanoparticles and different clusters.

### Example 2

Synthesis Au₂₅SG₁₈, Au₂₅SG₁₈ was synthesized from the as-prepared Au@SG clusters by core etching according to the literature procedure. (Shichibu, Y., et al. Small (2007) 3, 835; Habeeb, Muhammed, M. A., et al. Chem. Phys. Lett. (2007) 449, 186; Shibu, E. S., et al., J. Phys. Chem. C. (2008) 112, 12168; Habeeb Muhammed, M. A. et al., J. Phys. Chem. C. (2008) 112, 14324; the entire contents of each of the foregoing are incorporated herein by reference.) Briefly, the as-prepared Au@SG clusters were dissolved in water (25 mL). GSH was added (614 mg) and stirred at 55° C. The reaction was monitored by optical absorption spectroscopy. Heating was discontinued when the absorption features of Au₂₅SG₁₈ appeared in the UV/Vis spectrum. This typically took 12 hours (h) of heating. The solution was centrifuged and methanol was added to the supernatant to precipitate the cluster. The precipitate was dried to obtain Au₂₅SG₁₈ clusters in the powder form. The prepared Au₂₅SG₁₈ showed the characteristic UVN is (at 672 nm), FTIR, TEM, and NMR spectroscopic features described in the literature.

### Example 3

Synthesis of Au₂₃ clusters. The cluster was synthesized by interfacial etching. Au₂₅SG₁₈ clusters (10 mg) were dissolved in distilled water (10 mL). Octanethiol (8 times more than the amount of GSH present in Au₂₅SG₁₈) in toluene was mixed with the aqueous solution of Au₂₅SG₁₈ clusters. The biphasic mixture was stirred for 5 h at 25° C. During this time the absorption feature at 672 nm in the aqueous layer decreased and a new feature at 630 nm appeared. The aqueous and organic phases were separated and centrifuged. The aqueous layer contained Au₂₃SG₁₈ sub-nanoclusters (temporarily assigned as AuₓSG_{y}), while the residue was Au'SG polymer formed from the etched gold atoms. The organic layer was almost colorless and did not show any significant absorption feature. PAGE analysis of the aqueous layer showed only one fluorescent band, suggesting the presence of a single well-defined sub-nanocluster.

### Example 4

Synthesis of Au₃₃ clusters. The cluster was also synthesized by interfacial etching. Au₂₅SG₁₈ clusters (10 mg) were dissolved in distilled water (10 mL). Octanethiol (8 times more than the amount of GSH present in Au₂₅SG₁₈) in toluene was mixed with the aqueous solution of Au₂₅SG₁₈ clusters. The biphasic mixture was stirred for 1 h at 55° C. The organic layer, which was initially colorless, turned dark brown. The aqueous and organic phases were separated and centrifuged. The toluene layer displayed an absorption maximum of 709 nm, similar to that at 710 nm displayed by Au₃₃SG₂₂. After centrifugation, the aqueous layer contained only a deposit of Au^{I}SG polymer. The isolated sub-nanocluster was temporarily assigned the formula Auₓ(OT_{y}).

### Example 5

Synthesis of Au₂₂ clusters. The synthesis was carried out by single-phase etching. Au₂₅SG₁₈ clusters (10 mg) were dissolved in distilled water (10 mL). 3-Mercaptopropyl trimethoxysilane was added to the cluster solution (2 times more than the amount of GSH present in Au₂₅SG₁₈). The mixture was stirred for 7 h. During etching, the color of the aqueous layer became increasingly reddish compared with that of Au₂₅. The solution showed an absorption feature of 540 nm and the characteristic absorption features of Au₂₅ had disappeared completely. The solution was centrifuged and the supernatant separated from the insoluble gold thiolate. The absorption feature of the sub-nanoclusters is similar to that for Au₂₂(SG)₁₇, also at 540 nm. The isolated sub-nanocluster was temporarily assigned the formula Auₓ(MPTS)_{y}.

### Example 6

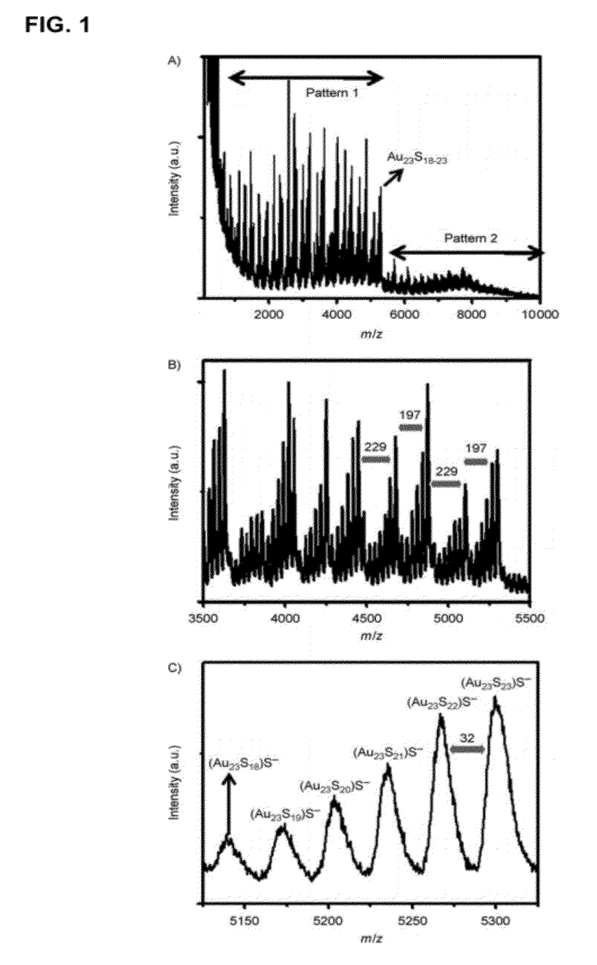

Mass Spectrometric Analysis of Gold Sub-Nanoclusters. The sub-nanoclusters of Examples 3-5 were examined using matrix desorption ionization (MALDI) mass spectrometry (Voyager DE Pro mass spectrometer of Applied Biosystems Inc). α-Cyano-4-hydroxycinnamic acid (CHCA) was used as the matrix. The spectrum was collected in the negative mode. The mass spectrum shows peaks with m/z values ranging from 100 to 10,000 (FIG. 1A). Peaks at low m/z regions are very intense with huge background signals when compared to those at higher m/z values. There is a pattern of peaks between m/z 1800 and 5300 and another pattern from m/z 5500 to 9000. The second set of peaks can be due to the clustering of ions detected in the lower m/z values. Clustering of clusters was observed in MALDI-MS studies of clusters. (Cyriac, J., et al., Chem. Phys. Lett. (2004) 390, 181, the entire contents of which are incorporated herein by reference.) The mass spectrum is composed of several groups of peaks with spacing of m/z 197 or 229 between the major peaks, as shown in FIG. 1B. This corresponds to the loss of Au or AuS. The m/z spacing between isolated peaks in each cluster of peaks is 32 on account of sulfur. These results are consistent with the earlier reports of laser-desorption mass spectrometry of gold clusters protected with thiolates. (see, e.g., Schaaff, T. G., Anal. Chem. (2004) 76, 6187, the entire contents of which are incorporated herein by reference.) Each bunch of peaks can be assigned as [AuₘSₙ]⁻. Since laser irradiation cleaves the S—C bond of the ligands, we can observe only peaks due to Auₘ clusters covered with S, and not the entire ligand. The last group of peaks of pattern 1 is due to Au₂₃S₁₈₋₂₃ (FIG. 1A). It is worth noting that after the peak due to Au₂₃, the intensity drops drastically. The major peak at m/z 5140 can be assigned to [(Au₂₃S₁₈)S]⁻; then addition of S leads to [(Au₂₃S₁₉)S]⁻, [(Au₂₃S₂₀)S]⁻, and so on (FIG. 1C). Such additions are observed in MALDI and laser desorption ionization (LDI). From these, we can tentatively assign a core of Au₂₃ for the AuₓSG_{y} cluster.

### Example 7

### FTIR Analysis of Gold Sub-Nanoclusters.

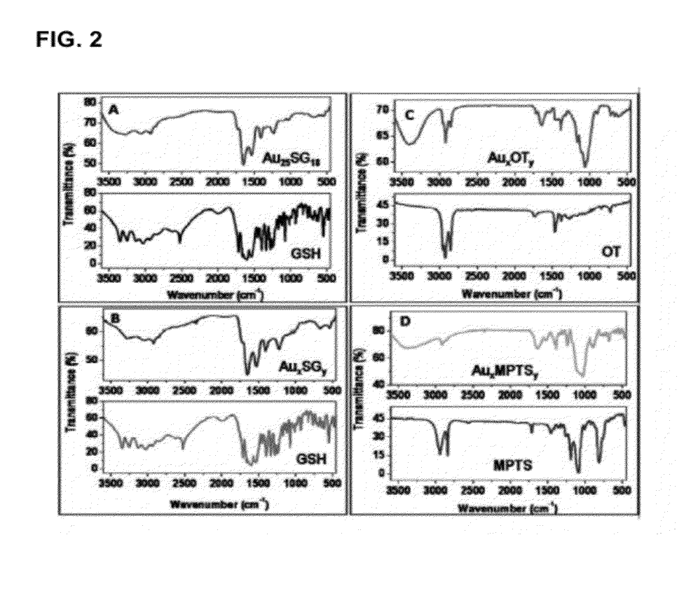

FTIR was carried out on the sub-nanoclusters of Examples 3-5. The FTIR spectra were measured using a Perkin-Elmer Spectrum One instrument. KBr crystals were used as the matrix for preparing the samples, which were at 5 wt % in KBr. FTIR spectra of the three clusters were compared with those of parent Au₂₅SG₁₈ and the ligands used for etching (FIG. 2A-D). FTIR spectra of the ligands bound on the cluster surface are less intense than the free ligands, owing to the fact that these ligands are linked to the cluster surface through covalent bonds. They are also distributed non-uniformly on the cluster surfaces, hence their vibrational features are masked to some extent. FTIR spectra of Au₂₅SG₁₈ and AuₓSG_{y} match exactly with each other and therefore it can be concluded that the AuₓSG_{y} clusters are protected completely with glutathione as in Au₂₅SG₁₈, which makes them water soluble. The peak at 2526 cm⁻¹ due to the —SH stretching of glutathione disappeared both in Au₂₅SG₁₈ and in AuₓSG_{y}, thereby suggesting the covalent bonding of glutathione with the cluster core through the thiolate link. The FTIR spectrum of AuₓSG_{y} shows features due to octanethiol. The peak at 2568 cm⁻¹ due to the —S—H stretching mode of octanethiol disappeared in AuₓOT_{y} confirming the covalent binding of octanethiol with the cluster core through the —SH group. The presence of OT on the cluster surface can also be confirmed by the large intensity of the —CH₂ stretching modes at 2846 and 2918 cm⁻¹. Since single-phase etching was carried out in water, 3-mercaptopropyl trimethoxysilane underwent hydrolysis to 3-mercaptopropyl trisilanol. FTIR spectra of AuₓMPTS_{y} showed features due to both 3-mercaptopropyl trisilanol and glutathione, which suggested that the cluster is protected with a mixed monolayer of 3-mercaptopropyl trimethoxysilanol and glutathione. The features due to 3-mercaptopropyl trisilanol were more significant compared with the glutathione features. The ligand protection of the clusters can now be summarized as follows.

### Example 8

Elemental Analysis of Gold Sub-Nanoclusters. Whereas AuₓSG_{y} is protected with glutathione, AuₓMPTS_{y} is protected with a mixed monolayer of 3-mercaptopropyl trisilanol and glutathione. AuₓOT_{y} is covered by octanethiol (all in thiolate form). To check the presence of gold and other elements, energy dispersive analysis of X-ray (EDAX) was carried out (data not shown) by drop-casting the aqueous solution of AuₓSG_{y} and solution of AuₓOT_{y} in toluene on indium-tin oxide (ITO) glass plates. Since AuₓMPTS_{y} was expected to have silicon, the EDAX measurements were carried out by pasting the powder of the cluster on a carbon tape. The elements present in the clusters were mapped. Whereas AuₓSG_{y} contained Au, C, N, O, and S, the elements present in AuₓMPTS_{y} were Au, C, N, O, S, and Si. The elements present in AuₓOT_{y} were Au, C, and S. Based on that information, the core and ligand protection of all three clusters are known. To assign chemical compositions to the clusters, elemental analyses (CHNS) were carried out. The results are given in the Table below. The molecular formulae of the clusters were found to be Au₂₂(MPTS)₁₀(SG)₇, Au₂₃(SG)₁₈, and Au₃₃(OT)₂₂.

| | | % of element | % of element | Molecular |
|---|---|---|---|---|
| Sample | Element | (Experimental) | (Calculated) | formula |
| AuₓMPTS_{y} | N | 03.85 | 03.68 | Au₂₂(MPTS)₁₀(SG)₇ |
| | C | 15.29 | 15.03 | |
| | H | 02.71 | 02.62 | |
| | S | 06.31 | 06.81 | |
| AuₓSGₓ | N | 07.75 | 07.53 | Au₂₃SG₁₈ |
| | C | 20.68 | 21.52 | |
| | H | 03.45 | 02.87 | |
| | S | 05.48 | 05.74 | |
| AuₓOT_{y} | N | 00.00 | 00.00 | Au₃₃OT₂₂ |
| | C | 22.01 | 21.78 | |
| | H | 04.15 | 03.86 | |
| | S | 07.18 | 07.26 | |

### Example 9

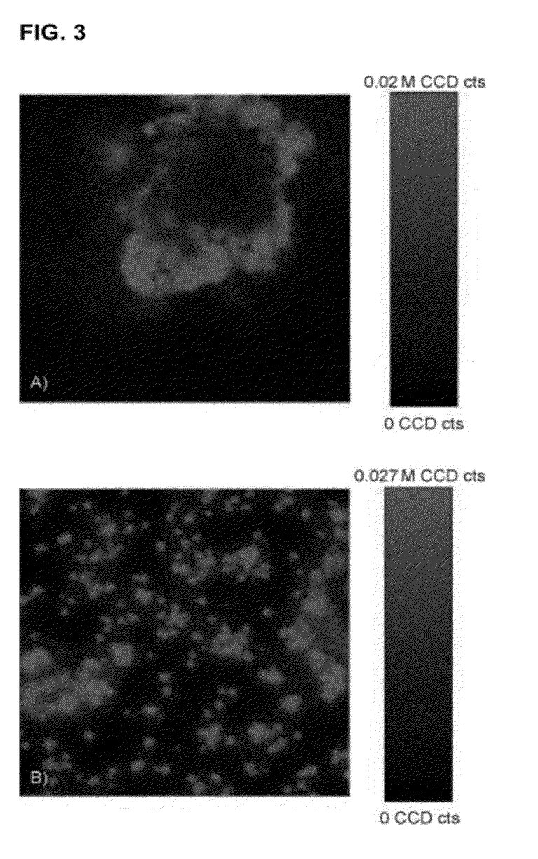

Luminescence of Gold Sub-Nanomolar Clusters. The sub-nanomolar clusters can be imaged using their inherent luminescence. Luminescence images of drop-casted Au₂₃ and Au₂₂ solid films were recorded using a confocal Raman spectrometer equipped with 532 nm excitation. The images in FIG. 3 show luminescence from Au₂₃ and Au₂₂ rich regions. Although there was luminescence from the red (bright) spots, there was no luminescence from the dark areas. The red spots are the islands of Au₂₂ or Au₂₃ clusters. These images show that the clusters are also luminescent in the solid state. Since Au₂₃ is a completely new cluster and is brightly luminescent and water soluble, further investigation of its properties were undertaken.

### Example 10

Conjugation of streptavidin with Au₂₃. Conjugation of streptavidin with glutathione-protected Au₂₃ was carried out using 1-ethyl-3-(3-dimethyl-aminopropyl)carbodiimide (EDC) as the coupling agent. EDC (25 μL of 10 mM) prepared in triply distilled water was added to a mixture of Au₂₃ (2 mg) and streptavidin (1 mg) in triply distilled water (1 mL). The mixture was stirred for 3 h. The streptavidin-coated Au₂₃ was subjected to dialysis for 2 d with a water change after every 6 h.

### Example 11

Imaging of Hepatoma Cells with Streptavidin-conjugated Au₂₃. Conjugated gold sub-nanoclusters of Example 10 were used to image human hepatoma cells (HepG2). These cancerous cells contain large amounts of biotin on their surfaces. Since biotin strongly binds with streptavidin, the cells can be imaged using the luminescence of the sub-nanoclusters in the form of a conjugate with streptavidin.

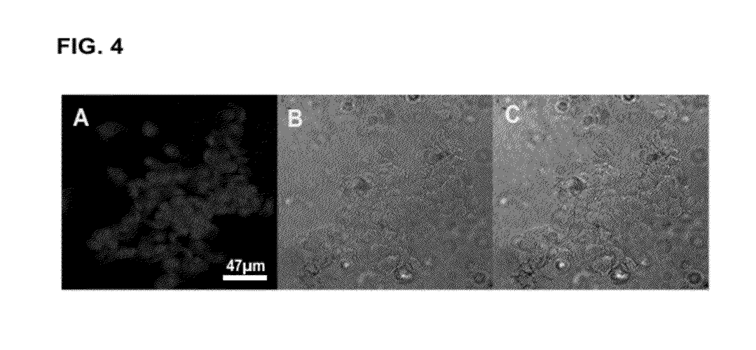

HepG2 cells were allowed to grow to 80% confluency starting with 2×10⁴ cells per well in 24-well tissue culture plates (in Dulbecco's Modified Eagle Medium (DMEM) supplemented with 10% fetal bovine serum (FBS) and incubated at 37° C. in humidified atmosphere containing 5% CO₂). For the imaging experiment, the growth medium was removed and the cells were washed twice with phosphate buffered saline (PBS) to remove the dye phenol red and various chemical reagents such as salts and glucose present in the growth medium. After this, the cells were fixed with 3% paraformaldehyde. Two-hundred microliters of a concentrated aqueous solution of streptavidin-conjugated Au₂₃ was added and incubated for 2 h at room temperature. After incubation the cells were washed several times with PBS to remove the unbound clusters and were imaged by confocal fluorescence microscopy. A very intense red luminescence was observed from the cells (see FIG. 4A-C). A control experiment was carried out to confirm the specificity of the streptavidin-biotin interaction. For this the fixed HepG2 cells were incubated with Au₂₃ clusters without any streptavidin conjugation for the same period. No luminescence was observed from the cells after washing (not shown). This experiment confirms that the specific interaction of streptavidin and biotin allows the cluster to stain the cells.

### Example 12

### Detection of Cu²⁺ Ions in an Aqueous Solution

The effect of fluorescence of Au₂₃ in the presence of various metal ions was studied. The ions selected were Au³⁺, Ag⁺, Cu²⁺, Ni²⁺, Ca²⁺, Mg²⁺, Na⁺, Pb²⁺, Hg²⁺, and Cd²⁺ as their nitrates or chlorides. Next, 50 ppm of the aqueous solutions of Au₂₃ was treated with metal ions so that the final concentration was 10 ppm, and the emissions of the clusters were measured immediately after the addition of ions. Au₂₃ was found to be reactive towards Cu²⁺. The emission of the cluster quenched significantly (from an intensity of 4×10⁶ to essentially zero). Although cluster emission enhanced a bit in the presence of Ag ions (from an intensity of 4×10⁶ to a little more than 5×10⁶), it remained unaltered in the presence of other metal ions.

### EQUIVALENTS

The present disclosure is not to be limited in terms of the particular embodiments described in this application. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All publications, patent applications, patents, and other references mentioned herein are expressly incorporated by reference in their entirety, to the same extent as if each were incorporated by reference individually. In case of conflict, the present specification, including definitions, will control.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A gold sub-nanocluster comprising a gold core and one or more thiolates bound to the gold core, wherein the gold core consists essentially of 23 gold atoms.

2. The gold sub-nanocluster of claim 1 wherein the one or more thiolates are selected from the group consisting of glutathione thiolate, 3-mercaptopropyl trimethoxy silane, octanethiolate and a mixture of any two or more thereof.

3. The gold sub-nanocluster of claim 1 wherein the one or more thiolates is glutathione thiolate.

4. The gold sub-nanocluster of claim 1 having the formula Au₂₃SG₁₈ wherein SG is glutathione thiolate.

5. The gold sub-nanocluster of claim 1 further comprising one or more fluorescent ligands selected from the group consisting of dansyl, FITC, green fluorescent protein, coumarin, fluorescein, and cyanine dyes.

6. The gold sub-nanocluster of claim 1 further comprising one or more amine or phosphine ligands.

7. A composition comprising an aqueous solution, an organic solution or a mixture thereof that comprises one or more gold sub-nanoclusters of claim 1.

8. The gold sub-nanocluster of claim 1, conjugated to a targeting molecule selected from the group consisting of a protein, a polynucleotide, or a ligand that binds to a protein or polynucleotide.

9. The gold sub-nanocluster of claim 8 wherein the targeting molecule is streptavidin, biotin, an antibody, folic acid, lactoferrin, transferrin, or tat protein.

10. A method comprising core etching one or more Au₂₅SG₁₈ sub-nanoclusters using a molar excess of thiol relative to the molar amount of SG, to provide one or more gold sub-nanoclusters, wherein SG is glutathione thiolate.

11. The method of claim 10 wherein the one or more Au₂₅SG₁₈ sub-nanoclusters are dissolved in an aqueous solution and the excess thiols are dissolved in an organic solvent that forms a two-phase system with the aqueous solution.

12. The method of claim 11 wherein the organic solvent is selected from the group consisting of toluene and xylene.

13. The method of claim 11 wherein the thiol is selected from the group consisting of glutathione, octanethiol, 3-mercaptopropyl trimethoxy silane thiol and a mixture of any two or more thereof.

14. The method of claim 10 wherein the gold sub-nanocluster produced consists essentially of 23 atoms of gold and one or more thiolates.

15. The method of claim 10 wherein the gold sub-nanocluster produced contains 22 or 33 gold atoms.

16. The method of claim 15 wherein the gold sub-nanoclusters produced have the formula Au₂₂(MPTS)₁₀(SG)₇ or Au₃₃OT₂₂.

17. A method comprising labeling a biological target with the conjugate of claim 8, and detecting the conjugate by luminescence.

18. The method of claim 17 wherein the biological target is a cancer cell.

19. The method of claim 17 wherein the luminescence is excited at a wavelength from about 400 to about 550 nm and the emission is detected at a wavelength from about 600 nm to about 800 nm.

20. A method comprising treating an aqueous sample to be tested for Cu²⁺ ions with an effective amount of gold sub-nanoclusters and measuring the decrease in luminescence of the treated sample, wherein the gold sub-nanoclusters each comprise a gold core and one or more thiolates bound to the gold core, wherein the gold core consists essentially of 23 gold atoms.
